(19) United States
(12) Patent Application Publication
(10) Pub. No.: US 2025/0059517 A1
(43) Pub. Date: Feb. 20, 2025

(54) MEDIA AND METHODS FOR MAKING AND MAINTAINING PORCINE PLURIPOTENT STEM CELLS

(71) Applicant: Recombinetics, Inc., Eagan, MN (US)

(72) Inventors: Abigail Sproull, Lino Lakes, MN (US); Ohad Gafni, Gedera (IL)

(21) Appl. No.: 18/807,650

(22) Filed: Aug. 16, 2024

Related U.S. Application Data
(60) Provisional application No. 63/533,574, filed on Aug. 18, 2023.

(51) Int. Cl. C12N 5/074; C12N 5/00; C12N 9/22; C12N 15/11

(52) U.S. Cl. CPC C12N 5/0696; C12N 5/0018; C12N 9/22; C12N 15/111; C12N 2310/20; C12N 2500/38; C12N 2501/16; C12N 2501/235; C12N 2501/415; C12N 2501/48; C12N 2501/602; C12N 2501/603; C12N 2501/604; C12N 2501/605; C12N 2501/606; C12N 2501/608; C12N 2506/02

(57) ABSTRACT

The present disclosure relates to methods for making porcine pluripotent stem cells. The present disclosure also relates to methods for gene-editing porcine pluripotent stem cells, generating animals from porcine pluripotent stem cells, and maintaining porcine pluripotent stem cells in culture over many passages. The present disclosure further relates to methods for making porcine induced pluripotent stem cells.

### CROSS-REFERENCE

This application claims the benefit of U.S. Provisional Application No. 63/533,574 filed Aug. 18, 2023, entitled “MEDIA AND METHODS FOR MAKING AND MAINTAINING PORCINE PLURIPOTENT STEM CELLS”, which is incorporated by reference herein in its entirety.

### BACKGROUND

Genetically modified pigs can be created through the process of cloning or somatic cell nuclear transfer (SCNT) in which a cell is edited and then the whole cell or just its nucleus is fused into an enucleated zygote to then develop into an embryo proper. Cloning and gene editing can be done with fibroblast lines, which can be edited once before needing rejuvenation; thus, the cost to create a gene edited animal can be significantly higher when multiple edits need to be made. Stem cells can be used in regenerative medicine and cell therapy space because, e.g., of their ability to differentiate into various cell and tissue types. They can also desirable due to their longevity and capacity for multiple genetic edits, thereby reducing cost for animal models of disease and xenotransplantation. There exists need for new and improved methods and compositions for the development of pluripotent porcine stem cell lines.

### SEQUENCE LISTING

The instant application contains a Sequence Listing which has been submitted electronically in XML format and is hereby incorporated by reference in its entirety. Said XML copy, created on Aug. 12, 2024, is named 53545-764.201.txt and is 8,833 bytes in size.

### SUMMARY

An aspect of the present disclosure is a method for making porcine pluripotent stem cells, the method comprising: obtaining porcine cells; culturing the porcine cells with porcine stem cell media, wherein the porcine stem cell media comprises one or more reagents selected from the group consisting of: a basal media, an L-glutamine source, a cell growth supplement, an antibiotic, a media stabilizer, an FGF protein, a SMAD regulator, an inhibitor of Lck, an inhibitor of Src, an inhibitor of Sik, a Wnt activator, a STAT3 activator, an antioxidant, ascorbic acid, and an inhibitor of apoptosis, thereby generating the porcine pluripotent stem cells. In some embodiments, the basal media comprises: DMEM, F12, Neurobasal™ media, N-2 supplement, or B27™ supplement. The L-glutamine source may comprise GlutaMAX™. The cell growth supplement may comprise amino acids. The amino acids may comprise non-essential amino acids. The antibiotic may comprise penicillin, streptomycin, gentamicin, kanamycin, or ampicillin. The media stabilizer may comprise bovine serum albumin and/or beta-mercaptoethanol. The SMAD regulator may comprise activin A. The Wnt activator may comprise CHIR99021. The STAT3 activator may comprise human leukemia inhibitory factor (LIF). The inhibitor of apoptosis may comprise SP600125. The antioxidant may comprise beta-mercaptoethanol, vitamin C, vitamin E, a carotenoid, ascorbate, glutathione, tocopherol, lipoic acid, ascorbic acid, or L-ascorbic acid.

In some embodiments, pluripotency of the porcine pluripotent stem cells is verified by way of alkaline phosphatase staining, pluripotent gene expression analysis, immunostaining, and/or cytogenetic analysis. The porcine pluripotent stem cells may maintain pluripotency over at least 5, 10, 15, 20, 25, 30, 35, 40, 45, 50, 55, 60, 65, 70, 75, 80, 85, 90, 95, or 100 or more passages. The porcine cells may comprise somatic cells. In some cases, the somatic cells comprise one or more of blood cells, skin cells, fibroblasts, and/or keratinocytes.

In some embodiments, the porcine somatic cells are transfected with one or more reprogramming factors. The one or more reprogramming factors may be encoded by one or more episomes. The one or more reprogramming factors may comprise one or more genes selected from the group consisting of: OCT3/4, SOX2, KLF4, C-MYC, LIN28, and NANOG. The porcine cells may comprise germ cells. The germ cells may be combined via in vitro fertilization (IVF) to produce an embryo. In some cases, the embryo is transferred to porcine stem cell media between days 1 and 12 post-fertilization. In some embodiments, porcine cells comprise cells obtained from non-IVF derived embryos. In some embodiments, porcine induced pluripotent stem cells derived from the porcine somatic cells are free of reprogramming factors.

In some embodiments, the method further comprises selecting morphologically stem cell-like colonies from the porcine stem cell media. The culturing may comprise incubating the porcine cells at a temperature from about 37° C. to about 38.5° C. with approximately 20% O2 and approximately 5% CO2. The porcine stem cell media may comprise mitotically inactivated feeder cells. The culturing may be feeder-free.

In some embodiments, the porcine pluripotent stem cells are gene-edited. In some cases, the porcine pluripotent stem cells are gene-edited via a CRISPR/Cas9 system, a CRISPR system, a TALEN system, a base editing system, a CRISPR/Cas9-derived RNA-guided engineered nuclease (RGEN) system, a zinc finger nuclease system, a prime editing system, or a variant thereof. The porcine pluripotent stem cells may be gene-edited at one or more target loci. The gene-editing may occur at the one or more target loci simultaneously. In some embodiments, one, two, three, four, five, six, seven, eight, nine, or ten targe loci are edited simultaneously. In some embodiments, the gene-editing occurs at one or more target loci in series. In some cases, one, two, three, four, five, six, seven, eight, nine, or ten target loci are edited in series. The porcine pluripotent stem cells may be transfected with gene-editing reagents. The porcine pluripotent stem cells may be treated to comprise gene-editing reagents. The porcine pluripotent stem cells may be transduced or otherwise manipulated via physical methods to comprise gene-editing reagents.

The method of the present disclosure may further comprise nuclear transfer cloning. In some embodiments, the porcine stem cells or the porcine pluripotent stem cells are fused with an enucleated embryo via nuclear transfer cloning. In some embodiments, the porcine stem cells or the porcine pluripotent stem cells are gene-edited prior to the nuclear transfer cloning. In some embodiments, the porcine stem cells or the porcine pluripotent stem cells are differentiated for 1, 2, 3, 4, or 5 days prior to the nuclear transfer cloning. In some embodiments, the porcine stem cells or the porcine pluripotent stem cells are contacted with a Wnt inhibitor prior to nuclear transfer cloning. In some embodiments, feeder cells are depleted over 1 or 2 hours. In some cases, the method of the present disclosure further comprises transfecting the porcine induced pluripotent stem cells with a vector encoding Retrotransposon Gag like 1 (RTL1). In some embodiments, the porcine induced pluripotent stem cells are free of reprogramming factors.

In some embodiments, the culturing technique by which the porcine stem cells are passaged comprises (i) allowing 80-90% confluence to be reached before dissociation with any enzymatic (like TrypLE) reagent to lift cells from the plate, (ii) lifting desired contents, (iii) placing in a conical tube, (iv) spinning down in a centrifuge, and (v) resuspending pellet at the desired split ratio to be replated. In some embodiments, the culturing technique by which the porcine stem cells are passaged comprises (i) allowing 80-90% confluence to be reached before dissociation with any non-enzymatic (like hypertonic citrate) reagent to lift cells from the plate, (ii) lifting desired contents, (iii) placing in a conical tube, (iv) spinning down in a centrifuge, and (v) resuspending pellet at the desired split ratio to be replated. In some embodiments, the method of the present disclosure comprises a culturing technique by which the porcine stem cells are frozen comprising (i) lifting and pelleting the cells, (ii) mixing with freezing media and placing in a cryovial, and (iii) storing at −80 or long-term storage in liquid nitrogen. In some embodiments, the method of the present disclosure further comprises a culturing technique by which the porcine stem cells are thawed comprising (i) removing vial from frozen storage, (ii) placing in an approximately 37° C. water bath or similar and allowing to thaw until a small ice pellet remains, (iii) moving to a biosafety cabinet, (iv) resuspending in growth media, (v) moving contents to a conical tube, (vi) spinning down, and (vii) resuspending resulting pellet at desired ratio before plating.

Another aspect of the present disclosure is an animal produced by the method of the present disclosure. A further aspect of the present disclosure is a cell line produced by the method of the present disclosure.

### BRIEF DESCRIPTION OF FIGURES

FIG. 1: A schematic of the process by which porcine pluripotent stem cells are created.

FIG. 2: An image of a representative porcine pluripotent stem cell colony at passage 3.

FIG. 3: Brightfield images of a representative progression during episomal reprogramming after 4 and 10 days after transfection.

FIG. 4: A schematic of the process by which the embryonic stem cell lines are derived.

FIG. 5: A representative progression of the process by which embryonic stem cells are derived.

FIG. 6: Brightfield images of day 19 embryos, denoted embryo 1 (E1) and embryo 2 (E2) cloned from a porcine embryonic stem cell at passage 17.

FIG. 7: Fluorescent images of staining of the pluripotency markers OCT4, SSEA1, TRA1-60, and SOX2 as well as brightfield images of staining for alkaline phosphatase activity.

FIG. 8: Karyotyping results are shown for porcine embryonic stem cells at passage 36 (left) and porcine induced pluripotent stem cells at passage 60 (right). Cytogenetic analysis was done on a representative porcine embryonic stem cell line and a representative porcine induced pluripotent line at both high and low passages.

FIG. 9: Pluripotent gene expression analysis is shown of both porcine embryonic stem cells (pESCs) and porcine induced pluripotent stem cells (piPSCs) (NANOG, SOX2, OCT4, and LIN28).

FIGS. 10A-B: Gels are shown depicting detection of targets using primer sequences. The results of FIG. 10A show detection of episomes and the results of FIG. 10B show detection of plasmids.

FIG. 11: Outlined here is a schematic of the steps performed to genetically modify the cell lines derived from the present disclosure.

FIGS. 12A-B: Stepwise edits in both pig ESCs and iPSCs using a CRISPR system to introduce donor DNA via homology-directed repair mechanisms showed similar efficiencies to the same edit in porcine fibroblasts.

FIG. 13: This graph depicts clonal editing efficiencies after clonal isolation has occurred (dots). Clonal efficiencies are similar to population efficiencies (shaded bars). Also included are the bolded line bars which show the percentage of the edited clonal population that were homozygous for the edit on both alleles.

### DETAILED DESCRIPTION

The subject matter of this application relates to compositions, methods, equipment, and systems for making porcine pluripotent stem cells. Various embodiments and examples of these compositions, methods, equipment, and systems are expressed below though still further similar embodiments are contemplated by the inventor. Each embodiment refers to one or more of various compositions, ingredients, components, steps, equipment, conditions, systems, and still other aspects. In addition, the inventor contemplates as part of this disclosure variations, combinations, and permutations of one of more such above-noted or following-described aspects beyond the specific embodiments set forth below, such as embodiments with fewer or more ingredients, steps and/or conditions that described in other embodiments, as well as combinations or permutations of one or more steps with one or more ingredients and/or one or more conditions.

In some embodiments, stem cell media used to derive and maintain both embryonic stem cells and induced pluripotent stem cells is the same. The media may comprise a mixture of one or more of basal medium, a serum-free supplement, vitamins, albumin, beta-mercaptoethanol, an L-glutamine supplement, a cell growth supplement, an N-2 supplement, glucose, amino acids, non-essential amino acids, an antibiotic, an antioxidant, ascorbic acid, a growth factor and/or a regulator thereof, a Wnt and/or a regulator thereof, an inhibitor of Lck, an inhibitor of Src, an inhibitor of Sik, a transcription factor and/or a regulator thereof, and/or a regulator of apoptosis. Antibiotics disclosed herein may include but are not limited to penicillin, streptomycin, gentamicin, kanamycin, and ampicillin. The antioxidant may comprise beta-mercaptoethanol, vitamin C, vitamin E, a carotenoid, ascorbate, glutathione, tocopherol, lipoic acid, ascorbic acid, or L-ascorbic acid. The media may optionally contain certain stabilizers to prevent oxidation and/or toxin buildup. Media stabilizers may include but are not limited to reducing agents such as beta-mercaptoethanol and/or protein stabilizers such as recombinant albumin, polyvinyl polymers, porcine dermal collagen, and bovine serum albumin. The media may be serum free. The media may comprise one or more of DMEM/F12, Neurobasal™ media, B27™ supplement, N-2 supplement, and L-glutamine source or supplement. The media may comprise regulators of apoptosis such as caspases, BH3 proteins, retinoic acid, JNK inhibitors, and protein or chemical inhibitors of apoptosis. JNK inhibitors may include but are not limited to SP600125, AS601245, and BMS986360. Vitamins may include but are not limited to riboflavin, retinoic acid, cyanocobalamin, thiamine, pyridoxine, biotin, pantothenate, nicotinamide, ascorbic acid, niacin, folic acid, vitamin B9, vitamin B6, vitamin B12, vitamin K, vitamin D, vitamin E, vitamin, A, vitamin C, and myo-inositol. The media may comprise regulators of factors such as SMADs, fibroblast growth factors (FGFs), Wnts, and/or STAT family proteins such as STAT3. A regulator of Wnt may be an activator of Wnt such as CHIR99021. An FGF protein may be FGF-basic. A regulator of a STAT family protein may be an activator of STAT3 such as human leukemia inhibitory factor (LIF). A regulator of SMAD may include but is not limited to activin A. In some embodiments, the L-glutamine source comprises GlutaMAX™.

Provided herein are the compositions of media and description of methods used to derive porcine pluripotent stem cells from embryos and non-pluripotent cells. The media comprises a 1:1 ratio of DMEM/F12 and Neurobasal supplemented with B27, N2, NEAA and P/S along with small molecule carriers, chemicals that prevent toxin buildup, regulators of energy metabolism, and cell proliferation as well as SMAD, WNT, STAT3, and apoptosis pathway regulators. Porcine pluripotent stem cells are cultured in this media and on an inactivated feeder layer. Together, this media and matrix is what drives the current disclosure's porcine pluripotent stem cells and their applications. In one aspect, the porcine stem cells are isolated from the inner cell mass of IVF-derived embryos at day 7 or from reprogrammed porcine embryonic fibroblasts. In a further aspect, the resulting porcine stem cells are pluripotent, edited, differentiated, and cloned.

The steps of the methods are not limited to a specific order. For example, one or more of the steps toward (i) determining effective amounts of the components within the porcine stem cell media; (ii) verification of pluripotency; (iii) editing of the porcine stem cells; (iv) differentiation of the porcine stem cells; and (v) cloning of the porcine stem cells or their derivatives can occur synchronously or in a stepwise fashion.

In one aspect, the porcine stem cell media comprises, or alternatively consists of, an effective amount of one or more of the following components: GlutaMAX, Bovine Serum Albumin (BSA), Non-Essential Amino Acids (NEAA), Penicillin-Streptomycin (P/S), FGF, Activin A, CHIR99021, Human LIF, Ascorbic Acid, and SP600125 or an equivalent thereof. Equivalents must impact the same overall pathway or effect the same mechanism as the original regulators such as apoptosis, WNT, SMAD, STAT3, and energy metabolism. In yet a further aspect, the porcine stem cell media is a modified N2B27 media or a mTeSR-based media.

In a further aspect, the feeder cells are mitotically inactivated (via mitomycin) mouse embryonic fibroblasts or can alternatively be replaced by another known organic matrix. Non-limiting examples of organic matrices include Vitronectin and Matrigel. In a yet further aspect, replacement of the feeder layer with an organic matrix and supplementation of the media with one or more of the following at effective concentrations: (i) Activin A, (ii) IWR1, and/or (iii) known small molecules that aid in the establishment of a feeder-free culture.

In another aspect, the embryo from which the pluripotent stem cells are isolated has been prepared by the method of in vitro fertilization, nuclear transfer cloning, parthenogenetic activation, natural or artificial insemination. Further, the pluripotent stem cells are isolated from a blastocyst, morula, or early-stage embryo. Yet further, the pluripotent stem cells are isolated from the original cell culture plate on which the embryos were seeded only after an effective amount of time when beginning to see colony formation.

In another aspect, the reprogrammed porcine fibroblast from which the pluripotent stem cells are isolated has been prepared by reprogramming with episomes. The method consists of (i) transfection of the effective amount of episomes namely, pCXLE-EGF, pCXLE-hOCT3/4 (OCT3/4), pCXLE-hSK (SOX2 & KLF4), and pCXLE-hMLN (C-MYC, LIN28 & NANOG); (ii) plating into a coated culture dish with the starting cell's media supplemented with a ROCK inhibitor; (iii) changing the media to porcine stem cell media at day 1 post-transfection; (iv) daily media changes; (v) and after an effective amount of time, manually picking of morphologically stem-like colonies. Yet further, the coated culture dish on which the transfected cells are plated is Vitronectin, gelatin, Matrigel, or another organic matrix.

After the selection process for both embryo-derived and induced pluripotent porcine stem cells they are maintained the same. The methods for maintenance of the porcine stem cells consists of, (i) allowing the culture to reach 80-90% confluence before passage; (ii) determining the ideal ratios to split the culture to allow 80-90% confluence to be achieved within 2-7 days, (iii) passaging by enzymatic or non-enzymatic means, (iv) pelleting and resuspending, and (v) seeding dropwise to the prepared plate. Yet further, the porcine stem cells are frozen by pelleting and resuspending in freeze media before being placed in a slow-freezing container and maintained at approximately −80° C.

Furthermore, the methods for genetically editing the cells, the method comprising, or alternatively consisting of, (i) dissociating to single cells or clumps of cells, (ii) delivering the genomic editing materials such as those that are CRISPR-based within each cell via transfection, (iii) plating the cells containing the editing materials onto a plate, (iv) determining editing efficiencies, (v) screening individual colonies, and (vi) expanding to create an edited population. The resulting population can then be used for downstream applications.

Further provided are any possible resulting differentiated cell types, cloned embryos, animals, and tissues created from the porcine stem cells of the current disclosure.

Any aspect or embodiment herein may be combined with any other aspect or embodiment as disclosed herein.

In one embodiment, a method of making a porcine pluripotent stem cell from one or more of the following:
(i) A porcine embryo derived via in vitro fertilization (IVF) at day 1-12 days post fertilization (dpf) of embryonic development.
(ii) A porcine embryo derived via SCNT at day 1-12 dpf of embryonic development.
(iii) A porcine embryo derived via natural means at day 1-12 dpf of embryonic development.
(iv) A porcine embryonic fibroblast.
(v) A porcine somatic cell.

In another embodiment, the porcine stem cell media can be effective with its current components at different concentrations such as, for example:
(i) N-2 supplement at concentrations between 0.1X and 2X;
(ii) B27™ supplement at concentrations between 0.1X and 2X;
(iii) GlutaMAX™ at concentrations between 0.1% and 2%;
(iv) NEAA at concentrations between 0.1% and 2%;
(v) Penicillin-Streptomycin at concentrations between 0.1% and 1%;
(vi) Bovine Serum Albumin at concentrations between 0 μg/mL and 200 μg/mL;
(vii) Fibroblast Growth Factor (FGF) at concentrations between 0 ng/mL and 50 ng/mL;
(viii) Activin A at concentrations between 0 ng/mL and 30 ng/ml;
(ix) CHIR99021 at concentrations between 0 μM and 5 μM;
(x) Human LIF at concentrations between 0 ng/mL and 20 ng/ml;
(xi) Ascorbic Acid at concentrations between 0 μg/mL and 200 μg/mL;
(xii) SP600125 at concentrations between 0 μM and 10 μM;
(xiii) Beta-mercaptoethanol at concentrations between 0 mM and 2 mM;
(xiv) IWR-1 at concentrations between 0 μM and 5 μM;
(xv) WH-4-023 at concentrations between 0 μM and 3 μM;
(xvi) SB590885 at concentrations between 0 μM and 2 μM; and/or
(xvii) PD0325901 at concentrations between 0 μM and 3 μM.

In another embodiment, the porcine stem cell media can be effective at maintaining pluripotency when other modulators of the same pathways controlled by the current components of porcine stem cell media are substituted or added. Pathways manipulated herein may include but are not limited to pathways related to:
(i) Energy metabolism, similar to the mechanisms of GlutaMAX™.
(ii) Cell proliferation, similar to the mechanisms of Ascorbic Acid and FGF.
(iii) SMAD, similar to the mechanisms of Activin A.
(iv) WNT, similar to the mechanisms of CHIR99021 via GSK3 or the mechanisms via Tankyrase inhibitors.
(v) STAT3, similar to the mechanisms of Human LIF.
(vi) Apoptosis, similar to the mechanisms of SP600125.
(vii) Lck, Src, and Sik similar to the mechanisms of WH-4-023.
(viii) MEK/ERK, such as mechanisms similar to B-Raf inhibitors and other controllers of proliferation, differentiation, and stress response.

In another embodiment, maintaining porcine pluripotent stem cells comprises one or more of:
(i) Maintaining a culture of porcine stem cells on a feeder layer and feeding with porcine stem cell media daily.
(ii) Maintaining a culture of porcine stem cells on an artificial matrix and feeding with a feeder-free porcine stem cell media daily.
(iii) Maintaining a culture of porcine stem cells on a feeder layer and feeding with porcine stem cell media every other day.
(iv) Maintaining a culture of porcine stem cells on an artificial matrix and feeding with a feeder-free porcine stem cell media every other day.
(v) Maintaining a culture at a density suitable for passage every 2-7 days.

In another embodiment, passaging porcine pluripotent stem cells comprises one or more of:
(i) Passaging by manually picking a single colony from a culture, considered selecting, and plating onto a new plate of feeders containing pre-equilibrated porcine stem cell media.
(ii) Passaging by dissociation with a non-enzymatic salt solution that does not intentionally disrupt the colony to single cells, but rather only lifts the entire colony from the plate to which it was attached.
(iii) Passaging by dissociation with an enzymatic solution that results in single cells.

In another embodiment, freezing the desired number of porcine pluripotent stem cells can occur in a freezing medium comprising:
(i) Porcine stem cell media and 10% DMSO.
(ii) Porcine stem cell media and the same volume of 2× freeze media made up of 80% Fetal Bovine Serum and 20% DMSO.
(iii) Any currently available cryogenic storage solution such as but not limited to CryoStor™.

In another embodiment, reprogramming of non-pluripotent cell types with episomes can be delivered either by, for example:
(i) Electroporation, such as with a Neon™ or BTX machine.
(ii) Transduction, such as a viral delivery of episomes.
(iii) Physical methods, such as with a laser to manually insert DNA into the cells.

In another embodiment, reprogramming with episomes can occur with varying quantities and at any combination of genomic material such as:
(i) 0 μg to 5 μg pCXLE-EGFP.
(ii) 0.1 μg to 10 μg pCXLE-hOCT3/4 (OCT3/4).
(iii) 0.1 μg to 10 μg pCXLE-hSK (SOX2 & KLF4).
(iv) 0.1 μg to 10 μg pCXLE-hMLN (C-MYC, LIN28 & NANOG).

In another embodiment, verification of pluripotency can be extended to include assays such as:
(i) Teratoma formation.
(ii) Embryoid Bodies.
(iii) Immunostaining for markers of pluripotency.

In another embodiment, the feeder layer used to maintain the porcine stem cells may comprise:
(i) Mitotically inactivated via mitomycin C mouse embryonic fibroblasts.
(ii) Mitotically inactivated via γ-irradiation mouse embryonic fibroblasts.
(iii) Modified mouse embryonic fibroblasts.
(iv) Fibroblast cells from any species.
(v) Epithelial cells from any species.
(vi) Mesenchymal cells from any species.

In another embodiment, gene-editing of cells as disclosed herein may comprise:
(i) Electroporation, such that the cells become porous and can take in the editing materials.
(ii) Transduction or by viral delivery in which the virus delivers the editing materials into the cells.
(iii) Physical means, such as with a laser in which editing materials are placed into the cells manually.
(iv) Transfection, such as with lipid nanoparticles or polyethylenimine (PEI).

Any aspect or embodiment herein may be combined with any other aspect or embodiment as disclosed herein.

### Definitions

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art. The terminology used herein is for the purpose of describing particular cases only and is not intended to be limiting.

As used herein, “a,” “an,” or “the” can mean one or more than one.

Herein the term “about” or “approximately” means within an acceptable error range for the particular value as determined by one of ordinary skill in the art, which will depend in part on how the value is measured or determined, e.g., the limitations of the measurement system. For example, “about” can mean within 1 or more than 1 standard deviation, per the practice in the art. Alternatively, “about” can mean a range of up to 20%, up to 15%, up to 10%, up to 5%, or up to 1% of a given value. Further, the term “about” when used in connection with a referenced numeric indication means the referenced numeric indication plus or minus up to 10% of that referenced numeric indication. For example, the language “about 50” covers the range of 45 to 55. Alternatively, particularly with respect to biological systems or processes, the term can mean within an order of magnitude, preferably within 5-fold, and more preferably within 2-fold, of a value. Where particular values are described in the application and claims, unless otherwise stated the term “about” meaning within an acceptable error range for the particular value should be assumed.

As used herein, the phrases “at least one”, “one or more”, and “and/or” are open-ended expressions that are both conjunctive and disjunctive in operation. For example, each of the expressions “at least one of A, B and C”, “at least one of A, B, or C”, “one or more of A, B, and C”, “one or more of A, B, or C” and “A, B, and/or C” mean A alone, B alone, C alone, A and B together, A and C together, B and C together, or A, B and C together.

As used herein, “or” may refer to “and”, “or,” or “and/or” and may be used both exclusively and inclusively. For example, the term “A or B” may refer to “A or B”, “A but not B”, “B but not A”, and “A and B”. In some cases, context may dictate a particular meaning.

Throughout this application, various embodiments may be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

As used herein, the word “include,” and its variants, is intended to be non-limiting, such that recitation of items in a list is not to the exclusion of other like items that may also be useful in the compositions and methods of this technology. Similarly, the terms “can” and “may” and their variants are intended to be non-limiting, such that recitation that an embodiment can or may comprise certain elements or features does not exclude other embodiments of the present technology that do not contain those elements or features.

The terms “comprise”, “comprising”, “contain,” “containing,” “including”, “includes”, “having”, “has”, “with”, or variants thereof as used in either the present disclosure and/or in the claims, are intended to be inclusive in a manner similar to the term “comprising.” Although the open-ended term “comprising” is used herein to describe and claim the disclosure, the present disclosure, or embodiments thereof, may alternatively be described using alternative terms such as “consisting of” or “consisting essentially of.”

The term “substantially” is meant to be a significant extent, for the most part; or essentially. In other words, the term substantially may mean nearly exact to the desired attribute or slightly different from the exact attribute. Substantially may be indistinguishable from the desired attribute. Substantially may be distinguishable from the desired attribute but the difference is unimportant or negligible.

The terms “increased”, “increasing”, or “increase” are used herein to generally mean an increase by a statically significant amount relative to a reference level. In some aspects, the terms “increased,” or “increase,” mean an increase of at least 10% as compared to a reference level, for example an increase of at least about 10%, at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level. Other examples of “increase” include an increase of at least 2-fold, at least 5 fold, at least 10-fold, at least 20-fold, at least 50-fold, at least 100-fold, at least 1000-fold or more as compared to a reference level.

The terms “decreased”, “decreasing”, or “decrease” are used herein generally to mean a decrease in a value relative to a reference level. In some aspects, “decreased” or “decrease” means a reduction by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (e.g., absent level or non-detectable level as compared to a reference level), or any decrease between 10-100% as compared to a reference level.

As used herein, the words “preferred” and “preferably” refer to embodiments of the technology that afford certain benefits, under certain circumstances. However, other embodiments may also be preferred, under the same or other circumstances. Furthermore, the recitation of one or more preferred embodiments does not imply that other embodiments are not useful and is not intended to exclude other embodiments from the scope of the technology.

“Maintain”, “Maintained”, or “Maintenance” when used in the context of a stem cell population means to cause the stem cells to continue to exist as a stem cell, that is, to have the ability to differentiate into any cell type of the three germ layers. The cells do this through the activity of certain genes and signaling pathways that work together to regulate the expression of key transcription factors that are responsible for the state of pluripotency.

“Maintainable” regarding a pluripotent stem cell line, meaning able to be maintained for at least 5, 10, 15, 20, 25, 30, 35, 40, 45, 50, 55, 60, 65, 70, 75, 80, 85, 90, 95 or 100 passages.

“Pluripotent” or “Pluripotency” means to have the ability to differentiate into all three germ layers within an embryo proper.

“Germ layers” refers to the three different lineages of cell fates including ectoderm, endoderm, and mesoderm which comprise the entirety of the embryo proper.

“Karyotypic stability” meaning that the chromosomal structures within the cells at early passages just after derivation and stabilization remain the same and are stable throughout the higher passages of the cell line.

“Obtain” or “Obtaining” refers to any means by which materials are collected by receiving from a third party, preparing internally, etc.

“Culturing” meaning the processes and methods by which the cells are maintained or prepared.

“Non-pluripotent” refers to the opposite of pluripotent cells in which the cells cannot differentiate into all three germ layers of the embryo proper. Most commonly, these cells have a specific cell fate or are completely differentiated to their ultimate cell type.

“Multipotent” refers to cells that can develop into more than one cell type within a single lineage but cannot develop into cells from all three germ layers like a pluripotent cell type. Thereby, they are more limited in their cell fates.

“Unipotent” refers to cells that can self-renew but do not have the capacity to differentiate into more than one cell type.

“Totipotent” refers to cells that are pluripotent and can differentiate into all three germ layers but can also contribute to extra-embryonic tissues such as the placenta. Typically, cells only remain totipotent during the first few cell divisions after fertilization.

“Passaging” meaning lifting a colony or many colonies from a plate and moving it/them to a new plate at a particular ratio of choice. Includes both picking manually with a pipette tip to transfer and chemically lifting the entire well/plate, pelleting via centrifugation, and resuspending at desired ratio to plate.

“Passage” or “Passages” referring to the number of times passaging, as described above, has occurred to a single cell line.

“Split ratio(s)” refers to the ratio of the original culture is used to continue the culture when passaging. An example being a split ratio of 1:2 meaning that in 2 new wells would be the same number of cells that used to be within a single (1) well.

“Morphologically stem-like” when used in the context of cells means that the cells cluster in a colony and said colony has defined edges and the cells within the colony are homogenous.

“Condition” or “Conditions” when discussing the cellular culture aspect of the disclosure encompasses all components that allow the cells to survive including, but not limited to, the media, the incubation temperature, the incubation gas levels, the incubation humidity levels, the matrix on which the cells reside, the plate format, etc.

“Transfection conditions” refers to the parameters that are set on the machine and the quantitative measurements of each component within the system during the transfection such as the number of cells or the amount of DNA added.

“Reprogram”, “Reprogrammed”, or “Reprogramming” regarding cells meaning the process by which a differentiated or somatic cell type is returned to a pluripotent state, thereby regaining their stem cell like qualities.

“Mitotically inactivated” or “Mitotically inactive” in referring to cells means that the cells have been treated in a certain way that prevents mitosis from occurring and thereby halting the cell cycle which results in non-dividing cells.

“Proliferate” meaning to multiply in numbers so in context the of cells means to continue to divide into more cells which then begins to take up more space in the plate or flask and then requires passaging.

“Dissociation” in a cell culture context meaning the process by which the cells are lifted from the plate. Can include either lifting to a single cell state or lifting as a colony or clumps of cells.

“Confluent” meaning the relative space being occupied within the culture dish.

“Non-IVF derived embryos” refers to embryos that are harvested from a naturally inseminated sow, or created via somatic cell nuclear transfer (SCNT), or made using in vitro fertilization (IVF) with intracellular sperm injection (ICSI), etc.

“Fetomaternal interface” meaning the highly specialized tissues that protect the fetus from immune-related injury.

“CRISPR system” refers to a RNA-protein interaction/mechanism that creates a double stranded break or single strand break (nick) at a desired site within the DNA to then be repaired by repair mechanisms either by non-homologous end joining, homology-directed repair with a donor template resulting in a modified sequence, or a single nucleotide converted to a specific base by an enzyme, for example a deaminase fused to a CRISPR system.

“Gene editing materials/reagents” collectively refer to the tools used for gene editing, minimally including a DNA modifying enzyme (ie. Cutting, nicking, deaminating, methylating, demethylating, etc.) directed to a specific location in the genome by interaction with a protein binding domain (TALENs and Zinc fingers) or an RNA guided protein domain (ie. Cas9, Cpf1, Cas12a, etc.) for example. These can be delivered to cells as plasmid expression cassettes, mRNAs/small RNAs, proteins, or protein/RNA complexes (RNPs), for example. Optionally, the materials could also include a repair template or a ‘homology directed repair” template. Gene editing materials could also include Prime editors—consisting of a DNA binding domain (ie. Cas9, Cpf1), a reverse transcription domain (either fused or separate), and prime-editing guide RNA (peg-RNA) that includes a reverse transcription primer and novel sequence to template a specific repair. Gene editing materials could also include base editors—consisting of a DNA binding domain (ie. Cas9, Cpf1) and a deaminase enzyme that converts one or a few bases in the base editing window from a “C” to a “T” (cytosine base editor, CBE) or an “A” to a “G” (adenine base editor, ABE).

“Homology-directed repair” refers to the type of repair mechanism by which a donor template with homologous sequences to both sides of the double stranded break or nick site is used to direct DNA repair and introduce any desired sequence to the break site. The template can be delivered as single strand DNA or double strand DNA by transfection or infection with viruses.

“Homozygous” meaning that the two alleles of a particular gene are the same.

“Homogeneous” when referring to a culture meaning of the same genetics, morphology, cell type, etc.

“Seeding” refers to the process by which a desired cell number is loaded into a cell culture plate. Accompanies the word density when referring to passaging and split ratios.

“Differentiate” or “Differentiated” when referring to stem cells meaning the cells are no longer pluripotent and their cell fate has been decided.

“Serial” or “Serially” when referring to the process of editing means the edits are done one after the other. So, a wild-type cell will be edited with a single edit, expanded, and then edited again so that the cells contain both edits and so on.

“Multiplex” or “Multiplexed” when referring to the process of editing details edits that are done simultaneously at a first loci and a second loci. A resulting cell from multiplex editing, from a single transfection or introduction of gene editing components, will contain edits at the first and second loci.

“Episome,” in context to the plasmids used to reprogram cells into induced pluripotent stem cells, refers to a DNA plasmid that is extrachromosomal and replicates autonomously, thereby remaining a part of eukaryotic genome without integration.

### EXAMPLES

### Example 1: Porcine Embryonic Stem Cell Derivation

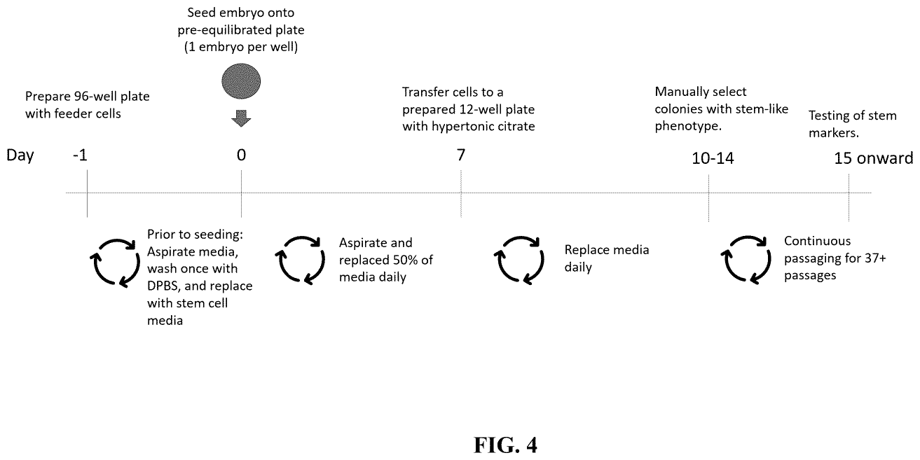

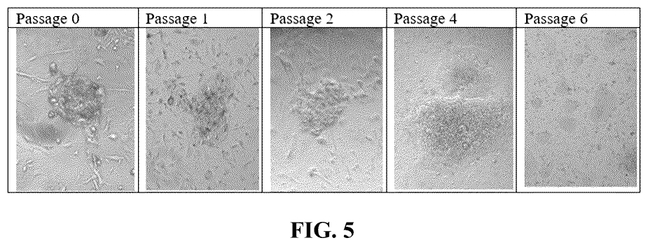

Individual day 6-7 embryos were manually transferred to a single well of a 96 well plate pre-equilibrated (in 37° C. incubator with 20% O2 and 5% CO2) plate with feeders containing 200 ul of porcine stem cell media per well. During the first week of culture, 100 ul of the media was carefully aspirated and replaced with 100 ul of fresh media daily. After the first week, cells were passaged to a 12 well plate pre-equilibrated plate with feeders and 1 mL of porcine stem cell media per well (FIG. 4). Each well was passaged individually to maintain a single embryo derived cell line. Hypertonic Citrate was used to passage the cells to not disrupt the formation of any immature colonies, however once colony-like structure was visualized (FIG. 5, passage 4) more dissociative or selective methods were used during passage to increase the number of colonies for expansion.

In an embodiment, collecting stem cell-like colonies after culturing in porcine stem cell media and passaging to a new plate is considered the first passage. In an embodiment, culturing porcine stem cells comprises growing cells on a gelatin coated plate. In some cases, the plate on which the cells are cultured is coated with a matrix. In certain cases, the matrix comprises gelatin. In some cases, the matrix comprises mitotically inactivated feeder cells.

### Example 2: Transfection of Reprogramming Tools Into Porcine Embryonic Fibroblasts

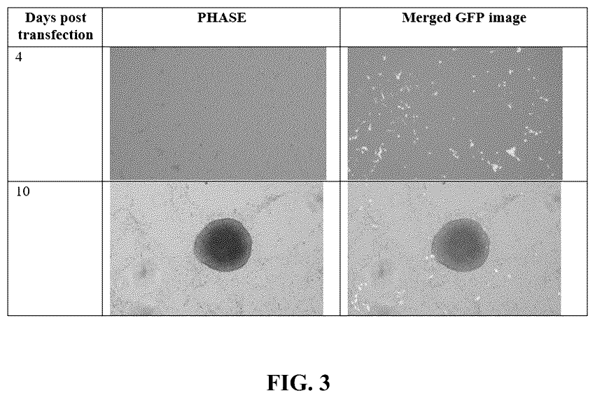

A low passage culture of porcine embryonic fibroblasts was allowed to come to 80-90% confluency before lifting to single cells with TrypLE, pelleted, resuspended, and counted. Aliquots of one million cells were pelleted before being transfected. 1 million pelleted cells were resuspended in approximately 100 μL of buffer formulated to ensure stability of restriction enzymes, and the episomes were added directly to the resuspension. The episomes that were added carried one or more of reprogramming factors known as Yamanaka factors. Those episomes were namely: pCXLE-hOCT3/4 (OCT3/4), pCXLE-hSK (SOX2 & KLF4), and pCXLE-hMLN (C-MYC, LIN28 & NANOG) and 2.5 μg of each was added to the resuspension. In some embodiments, 1 μg of the episome pCXLE-EGFP was also added to the resuspension to visualize the success of the transfection. After the transfection was completed, cells were plated into a 100 μg/mL vitronectin coated 10 cm plate with mouse conditioned embryonic fibroblast (MEF) media and 10 uM ROCK inhibitor that was pre-equilibrated in a 37° C. incubator with 5% CO₂ and 20% O₂. Media was changed the next day to porcine stem cell media and fluorescence was visualized to ensure the uptake of the vectors. Media was changed daily, and colony visualization occurred by day 10 (FIG. 3). Those colonies were manually picked out of the derivation plate and individually placed into a single well of a 12-well plate to allow expansion and establishment of a cell line.

In some embodiments, culturing of the porcine stem cells comprised (i) allowing 80-90% confluence to be reached before dissociation with any enzymatic (like TrypLE) reagent to lift cells from the plate, (ii) lifting desired contents, (iii) placing in a conical tube, (iv) spinning down in a centrifuge, and (v) resuspending pellet at the desired split ratio to be replated. In some embodiments, culturing of the porcine stem cells comprised (i) allowing 80-90% confluence to be reached before dissociation with any non-enzymatic (i.e. hypertonic citrate) reagent to lift cells from the plate, (ii) lifting desired contents, (iii) placing in a conical tube, (iv) spinning down in a centrifuge, and (v) resuspending pellet at the desired split ratio to be replated. In some embodiments, the culturing comprised freezing the porcine stem cells via (i) lifting and pelleting the cells, (ii) mixing with freezing media and placing in a cryovial, and (iii) storing at −80 or long-term storage in liquid nitrogen. In some embodiments, the culturing comprised thawing the porcine stem cells via (i) removing a vial of frozen cells from frozen storage, (ii) placing in an approximately 37° C. water bath or similar and allowing to thaw until a small ice pellet remains, (iii) moving to a biosafety cabinet, (iv) resuspending in growth media, (v) moving contents to a conical tube, (vi) spinning down, and (vii) resuspending resulting pellet at desired ratio before plating.

### Example 3: Long-term Culture of Porcine Pluripotent Stem Cells

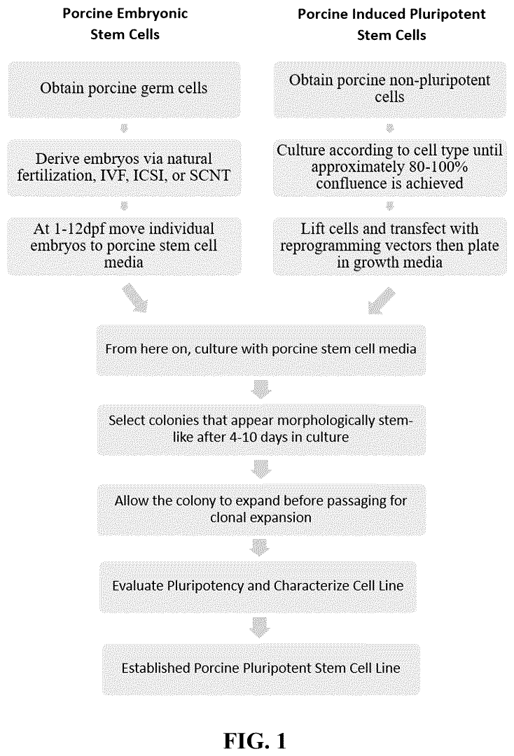

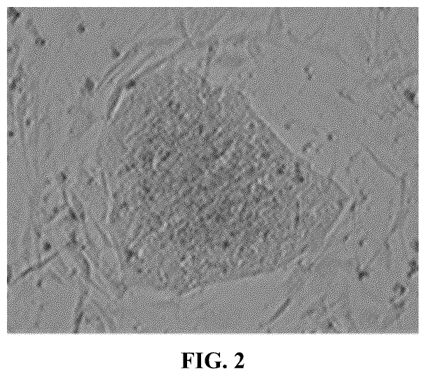

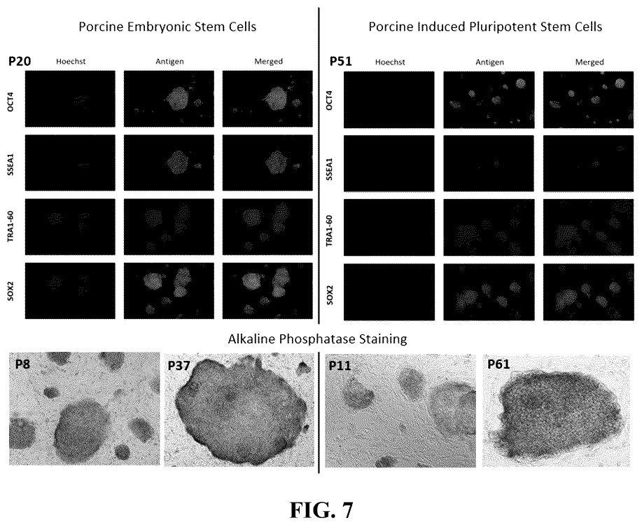

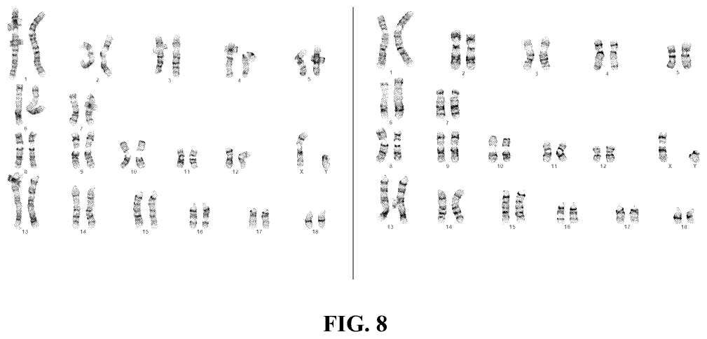

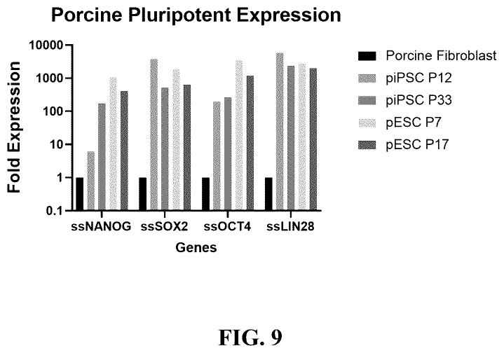

Porcine embryonic stem cells and porcine induced pluripotent stem cells, once established, remained pluripotent and unchanged at least into passages thirty and sixty, respectively (FIG. 7). Porcine pluripotent stem cell lines showed a round colony with defined edges (FIG. 2). Once the culture reached homogeneity, the cells were passaged every 3-4 days (FIG. 1). Porcine pluripotent stem cells were positive for alkaline phosphatase (AP) staining and one or more proteins associated with pluripotency including nuclear OCT4 and SOX2 and cytoplasmic SSEA1 and TRA1-60. Porcine embryonic stem cells and porcine induced pluripotent stem cells were karyotyped at both low and high passages to show their chromosomal stability (FIG. 8). Gene expression analysis for characteristic pluripotent genes also showed the stability of these cell lines to remain pluripotent over the course of many passages (FIG. 9).

FIG. 7 shows that alkaline phosphatase activity was increased in stem cell populations in comparison to differentiated cell types, in both porcine embryonic stem cells (left) and porcine induced pluripotent stem cells (right). Porcine embryonic stem cells pluripotency marker staining is shown at passage 20 (P20) and alkaline phosphatase staining at passage 8 (P8) and passage 37 (P37). Porcine induced pluripotent stem cells pluripotency marker staining is shown at passage 51 (P51) and alkaline phosphatase staining at passage 11 (P11) and passage 61 (P61).

The ability for the long-term culture of these cell types is due to the composition of the porcine stem cell media as well as the culturing technique that allows for continuous passages to occur. The porcine stem cell media comprised one or more of the following: CHIR99021, Activin A, FGF, LIF, and Ascorbic Acid. However, it is unique in that the composition has been able to both derive and maintain porcine stem cells from different sources. The sources include but are not limited to day 7 in vitro fertilization (IVF)-derived embryos and porcine embryonic fibroblasts. The media composition remained unchanged from the point of derivation to the highest passage tested which is a testament to its durability and flexibility. In some embodiments, hypertonic citrate was used to maintain stemness and stability of cells.

### Example 4: Cloning of Porcine Embryonic Stem Cell and Porcine Induced Pluripotent Stem Cells

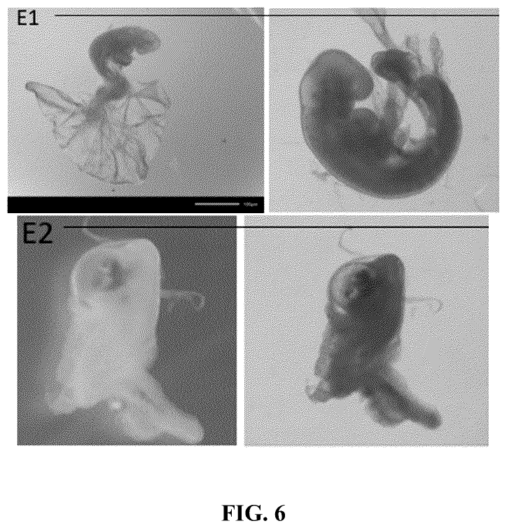

In some embodiments, porcine embryonic stem cells were cloned via nuclear transfer techniques, and embryonic day 19 fetuses were seen (FIG. 6). In some embodiments, porcine pluripotent stem cells were cloned via nuclear transfer techniques. In some embodiments, anti-apoptotic agents were used to allow for increased efficiency of the cloning process before embryo transfer. In some embodiments, exogeneous expression of porcine imprinted Retrotransposon Gag like 1 (RTL1) gene was used to assist in cloning efficiency. In some cases, cells were transfected prior to cloning with a transient plasmid that carries the RTL1 gene and allows it to be overexpressed within the cell. In some cases, this expression continued after embryonic development.

In an embodiment, the porcine stem cells undergo a period of differentiation 1-5 days before cloning. In an embodiment, the porcine stem cells are isolated or derived from the matrix on which they are cultured, pelleted and resuspended in a maturation media, and fused to an enucleated oocyte. In an embodiment, the porcine stem cells are transfected with a vector encoding retrotransposon gag like 1 (RTL1) to aid with cloning efficiency. In an embodiment, a Wnt inhibitor is added to the maturation media. Maturation media may be used to prepare cells for cloning. In some embodiments, anti-apoptotic agents are used to increase efficiency of cloning. Porcine stem cells or porcine pluripotent stem cells as described herein may be utilized for cloning techniques as described herein. Porcine stem cells or porcine pluripotent stem cells as described herein may be utilized for gene-editing. Gene-editing of the porcine stem cells or porcine pluripotent stem cells may be carried out before the gene-edited porcine stem cells or gene-edited porcine pluripotent stem cells are utilized for cloning techniques as described herein.

As shown in FIG. 8, cytogenetic analysis was performed on twenty G-banded metaphase cells from pig embryonic stem cell line at passage 36 (left) and on twenty G-banded metaphase cells from pig induced pluripotent cell line at passage 60 (right). Nineteen cells demonstrated a normal male karyotype.

An embryo, or many embryos, tissues, a pig or many pigs resulting from this cloning and transfer process may have many applications. Examples include being used as large animal disease or drug screening models, breeders for favorable livestock traits, a sustainable way to meet increasing global food demands, as a biological incubator for organs, tissues, and cells with a destination of xenotransplantation, or as a biological manufacturer of blood, proteins, viruses, living organisms, and serums that will be used to create a biologic medication.

### Example 5: Gene Editing of Porcine Embryonic Stem Cell and Porcine Induced Pluripotent Stem Cells

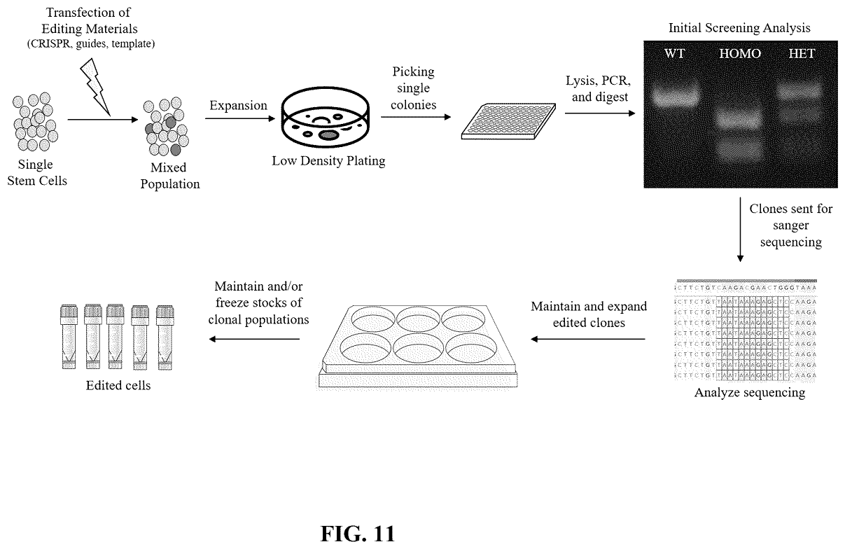

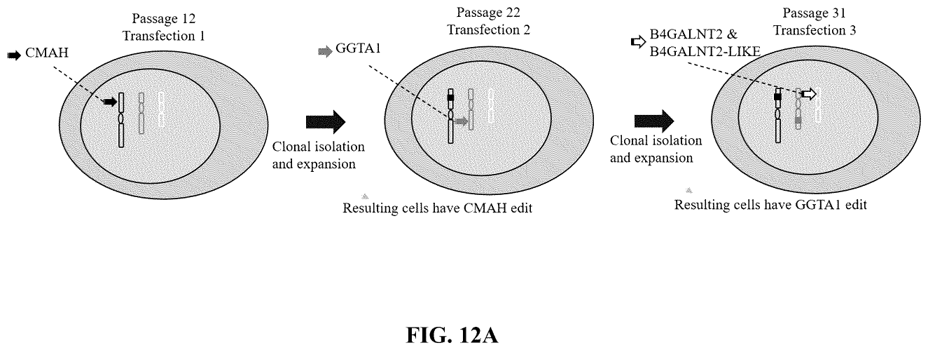

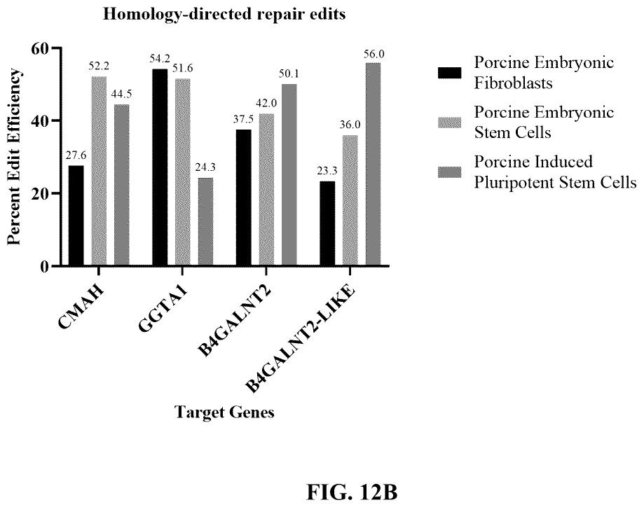

In an embodiment, porcine embryonic stem cell and porcine induced pluripotent stem cells were edited with the same sequence of edits done in a fibroblast line so that efficiencies could be compared. The porcine pluripotent stem cell lines of the present disclosure maintained pluripotency and continued to survive over many passages (FIG. 7-9) which allowed for more time to edit without needing to rejuvenate. It is of note however that the porcine embryonic fibroblasts may be transfected once at a low passage before needing to be rejuvenated, whereas the stem cells may be transfected multiple times, serially (FIG. 12A), and well into later passages. The transfection conditions and editing methods (FIG. 11) may be kept constant across cell lines and in some cases the editing efficiency for each of the genes may be higher than or around the same percentage of editing efficiency as shown with homology-directed repair (FIG. 12B).

Of note, in some embodiments, gene editing occurred serially as described in FIG. 12B except for editing of B4GALNT2 and the B4GALNT2-LIKE which occurred simultaneously within the same transfection.

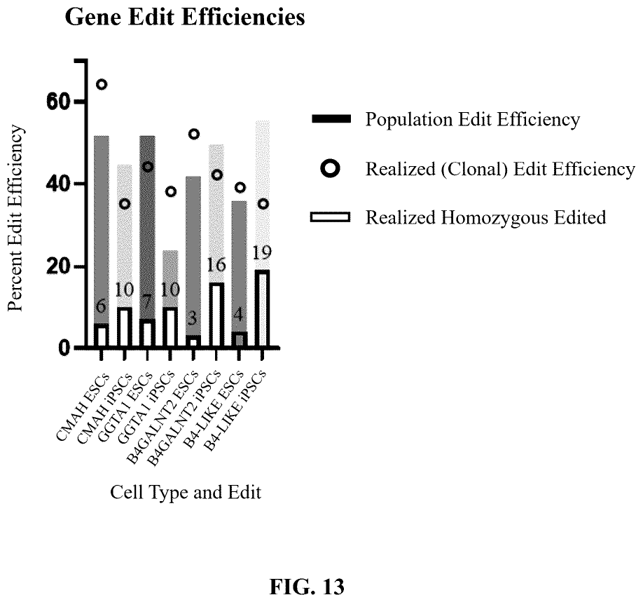

Once a cell population was edited, clones were isolated to obtain a homogenous cell line, which allowed for quantification of the realized edit efficiencies (FIG. 13) after each round of edits. Editing was done with a CRISPR delivery of editing reagents using a Neon™ transfection system but could also be effective using any type of electroporation method such as BTX, transduction, etc. Other gene editing delivery systems can also be utilized as best suited to the edit of interest.

In some embodiments, CRISPR reagents comprise Cas9 protein and one or more guide RNA. In some embodiments, CRISPR reagents further comprise one or more repair templates. In some embodiments, cells are gene-edited via a CRISPR/Cas9 system, a CRISPR system, a TALEN system, a base editing system, a CRISPR/Cas9-derived RNA-guided engineered nuclease (RGEN) system, a zinc finger nuclease system, a prime editing system, or a variant thereof

The first two edits were done serially in that one homozygous edit was completed before the next transfection for a second edit. However, in one embodiment a multiplexing edit was performed on the third editing round since B4GALNT2 and B4GALNT2-LIKE are separate loci, requiring two separate guide RNAs and donors (FIG. 12A).

In some embodiments, gene editing is performed serially. In some embodiments, multiple chromosomal loci or gene targets are edited serially. In some embodiments, multiple chromosomal loci or gene targets are edited at the same time via multiplex editing. One, two, three, four, five, six, seven, eight, nine, or ten chromosomal loci or gene targets may be edited in series. One, two, three, four, five, six, seven, eight, nine, or ten chromosomal loci or gene targets may be edited at the same time. Chromosomal loci as utilized herein may refer to distinct genomic or extra-genomic sequences.

FIG. 11 shows a schematic of the process of gene editing to establish a cell line containing the edit of interest. First, a population of cells was transfected with the required editing materials. Then the cells were plated and allowed to expand to become a mixed population, which can then be screened for the edit. This initial population screening allows for the determination of how many colonies to pick in order to obtain a homozygous cell line. The mixed population of cells were low-density plated, picked, and screened (PCR, digest, analysis) before sending for sanger sequencing. Once sequencing results were received and analyzed, the edited clones were expanded to create an edited cell line stock.

FIGS. 12A and 12B show (12A) a schematic of the serial (CMAH and GGTA1) and multiplex (B4GALNT2 and B4GALNT2-LIKE) editing conducted with stem cell lines at specific passages and (12B) an associated graph describing editing efficiencies via homology-directed repair mechanisms of different xenotransplantation-enabling genes (CMAH, GGTA1, B4GALNT2 and B4GALNT2-LIKE) within the porcine embryonic stem cell (light gray) and porcine induced pluripotent stem cell (dark gray) lines in comparison with the editing efficiencies in porcine embryonic fibroblast lines (black).

FIG. 13 shows a graph of realized edit efficiency when clones were picked from a given population (represented by dots) graphed atop the population editing efficiency (shaded bars). Each of the shorter bars with the bolded outline shows the percentage of the total picked clonal population that was shown to be homozygous for the edit of interest (labeled with the percent on top of the bar). Of note, the realized homozygous edit efficiency of the multiplexed edit of B4GALNT2 and B4GALNT2-LIKE for the ESCs is 2% of the picked clonal population and for the iPSCs is 11% of the picked clonal population.

### Example 6: Additional Growth Factors for Supporting Growth of Stem Cells

In some embodiments, additional growth factors were added to support the growth of porcine stem cells. In some embodiments, growth factors are provided by a feeder layer or by the addition of supplemental growth factors, which can be added to the media to support porcine stem cell culture. A feeder layer is visible in stem cell images disclosed herein (FIGS. 2, 3, 5, 7). The feeder layer may be comprised of but is not limited to mitotically inactivated mouse embryonic fibroblast cells. In some embodiments, growth factor supplementation may replace the need for a layer of feeder cells in cell culture. In some embodiments, cell culture techniques as disclosed herein may be feeder free.

### Example 7: Media Composition for Stem Cell Culture

In some embodiments, stem cell media used to derive and maintain both embryonic stem cells and induced pluripotent stem cells is the same. The media may comprise a mixture of one or more of basal medium, a serum-free supplement, vitamins, albumin, beta-mercaptoethanol, an L-glutamine supplement, a cell growth supplement, an N-2 supplement, glucose, amino acids, non-essential amino acids, an antibiotic, an antioxidant, ascorbic acid, a growth factor and/or a regulator thereof, a Wnt and/or a regulator thereof, an inhibitor of Lck, an inhibitor of Src, an inhibitor of Sik, a transcription factor and/or a regulator thereof, and/or a regulator of apoptosis. Antibiotics disclosed herein may include but are not limited to penicillin, streptomycin, gentamicin, kanamycin, and ampicillin. The antioxidant may comprise beta-mercaptoethanol, vitamin C, vitamin E, a carotenoid, ascorbate, glutathione, tocopherol, lipoic acid, ascorbic acid, or L-ascorbic acid. The media may optionally contain certain stabilizers to prevent oxidation and/or toxin buildup. Media stabilizers may include but are not limited to reducing agents such as beta-mercaptoethanol and/or protein stabilizers such as recombinant albumin, polyvinyl polymers, porcine dermal collagen, and bovine serum albumin. The media may be serum free. The media may comprise one or more of DMEM/F12, Neurobasal™ media, B27™ supplement, N-2 supplement, and L-glutamine source or supplement. The media may comprise regulators of apoptosis such as caspases, BH3 proteins, retinoic acid, JNK inhibitors, and protein or chemical inhibitors of apoptosis. JNK inhibitors may include but are not limited to SP600125, AS601245, and BMS986360. Vitamins may include but are not limited to riboflavin, retinoic acid, cyanocobalamin, thiamine, pyridoxine, biotin, pantothenate, nicotinamide, ascorbic acid, niacin, folic acid, vitamin B9, vitamin B6, vitamin B12, vitamin K, vitamin D, vitamin E, vitamin, A, vitamin C, and myo-inositol. The media may comprise regulators of factors such as SMADs, fibroblast growth factors (FGFs), Wnts, and/or STAT family proteins such as STAT3. A regulator of Wnt may be an activator of Wnt such as CHIR99021. An FGF protein may be FGF-basic. A regulator of a STAT family protein may be an activator of STAT3 such as human leukemia inhibitory factor (LIF). A regulator of SMAD may include but is not limited to activin A. In some embodiments, the L-glutamine source comprises GlutaMAX™.

In an embodiment, the media may comprise a 1:1 ratio of DMEM/F12 and Neurobasal™ supplemented with 1X of both B27™ and N-2 supplement and 1% of both NEAA and penicillin/streptomycin (P/S) along with small molecule carriers (50 ug/mL bovine serum albumin), chemicals that prevent toxin buildup (0.1 mM beta-mercaptoethanol), regulators of energy metabolism (1X L-glutamine supplement), and cell proliferation (20 ng/mL FGF and 50 ug/mL ascorbic acid) as well as SMAD (10 ng/ml Activin A), Wnt (1 uM CHIR99021), STAT3 (10 ng/ml human LIF), and apoptosis (5 uM SP600125) pathway regulators. Many of the associated regulators may not be required to derive and maintain porcine stem cells and could be useful at different concentrations within the media.

### Example 8: Evaluating Episomal Vectors

The induced pluripotent stem cells were reprogrammed using episomes. In some embodiments, the episomes containing one or more human Yamanaka factors were transiently expressed. In some embodiments, pluripotent gene expression of porcine specific markers was detected (FIG. 9) in the stem cells. In some cases, continuous passaging of the induced pluripotent stem cells reduced the amount of human gene expression via episomes. To quantify the amount of episomes still present in the induced pluripotent stem cells, the number of episomal backbone copies was tested as well as the expression of each individual human gene vector to determine which of the episomes still resided in the cells.

In some embodiments, the stem cells without episomes are selected for and expanded to create a transgene-free population of pluripotent stem cells. In some embodiments, the porcine induced pluripotent stem cells of the present disclosure do not comprise reprogramming factors. In some embodiments, the porcine induced pluripotent stem cells maintain stemness and/or pluripotency without comprising exogenous reprogramming factors. In some embodiments, the porcine induced pluripotent stem cells maintain stemness and/or pluripotency in the absence of the episomes.

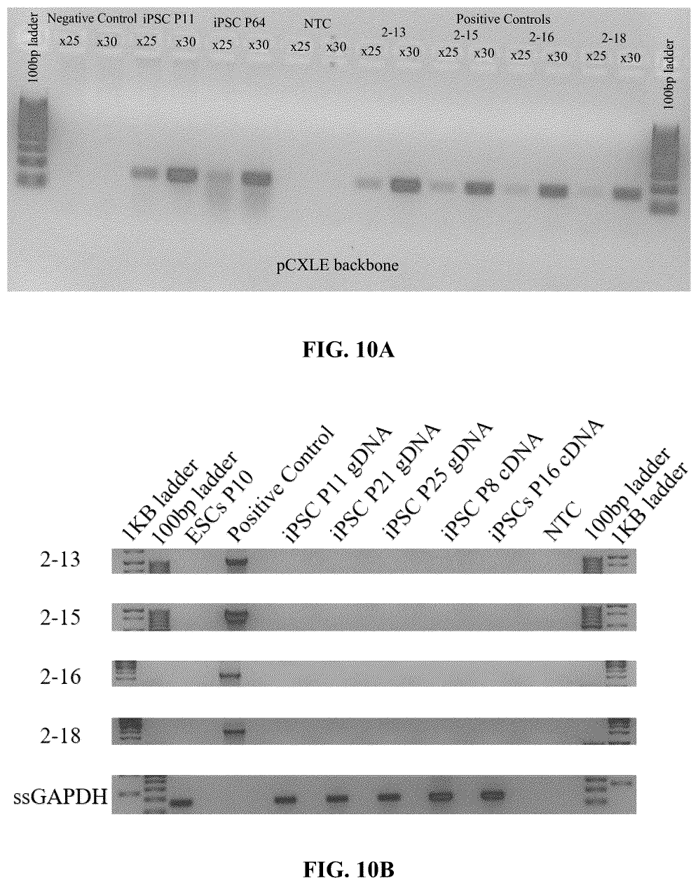

As shown in FIG. 10A, primer sequences were used to detect the presence of episomes within induced pluripotent stem cells. PCR was used for the amplification of the episome backbone (pCXLE) within 20 ng of DNA and 0.06 pg of episome DNA. In some cases, it was assumed that one copy of plasmid per cell was used to reprogram the non-pluripotent cells. Annotations of the PCR from left to right are as follows: negative control of wild-type porcine tail DNA at 25 cycles (×25) and 30 cycles (×30), then gDNA from an induced pluripotent stem cell line at passage 11 again with 25 cycles and 30 cycles, then the same porcine induced pluripotent stem cell line at passage 64 (×25 & ×30), followed by no template controls, then all four episomes serving as positive controls (×25 & ×30). The next gel shown (FIG. 10B) is of the detection of each individual plasmid within 20 ng of gDNA or 1 ng of cDNA. Primers used to detect individual plasmids contained a forward primer on one of the expression genes and a reverse primers on the conserved region of each episome abbreviated WPRE.

Table 1 below provides sequences of primers disclosed herein.

TABLE 1
Primer sequences

| Primer | Sequence |
|---|---|
| pCXLE Forward | AGGGCAGGAGTGATGTAACT (SEQ ID NO: 1) |
| pCXLE Reverse | GTTGGGAGGACGAAAATGGT (SEQ ID NO: 2) |
| 2-13 Forward | GAAAGAGAAAGCGAACCAGT (SEQ ID NO: 3) |
| 2-15 Forward | AACTACAACAGCCACAACGT (SEQ ID NO: 4) |
| 2-16 Forward | GTTCAACGATCTCCTGGACC (SEQ ID NO: 5) |
| 2-18 Forward | AAACGCAGATCCAAAGGAGA (SEQ ID NO: 6) |
| WPRE Reverse | AGGGAGATCCGACTCGTCTG (SEQ ID NO: 7) |
| ssGAPDH | GCCATCACTGCCACCCAGAA (SEQ ID NO: 8) |
| Forward | |
| ssGAPDH | GCCAGTGAGCTTCCCGTTGA (SEQ ID NO: 9) |
| Reverse | |

1. A method for making porcine pluripotent stem cells, the method comprising:
a) obtaining porcine cells;
b) culturing the porcine cells with porcine stem cell media, wherein the porcine stem cell media comprises one or more reagents selected from the group consisting of: a basal media, an L-glutamine source, a cell growth supplement, an antibiotic, a media stabilizer, an FGF protein, a SMAD regulator, an inhibitor of Lck, an inhibitor of Src, an inhibitor of Sik, a Wnt activator, a STAT3 activator, an antioxidant, ascorbic acid, and an inhibitor of apoptosis, thereby generating the porcine pluripotent stem cells.

2-7. (canceled)

8. The method of claim 1, wherein the SMAD regulator comprises activin A.

9. The method of claim 1, wherein the Wnt activator comprises CHIR99021.

10. The method of claim 1, wherein the STAT3 activator is human leukemia inhibitory factor (LIF).

11. (canceled)

12. The method of claim 1, wherein the inhibitor of apoptosis comprises SP600125.

13. (canceled)

14. The method of claim 1, wherein the porcine pluripotent stem cells maintain pluripotency over at least 5, 10, 15, 20, 25, 30, 35, 40, 45, 50, 55, 60, 65, 70, 75, 80, 85, 90, 95, or 100 or more passages.

15. The method of claim 1, wherein the porcine cells comprise somatic cells.

16. (canceled)

17. The method of claim 15, wherein the porcine somatic cells are transfected with one or more reprogramming factors.

18. The method of claim 17, wherein the one or more reprogramming factors are encoded by one or more episomes.

19. The method of claim 17, wherein the one or more reprogramming factors comprise one or more genes selected from the group consisting of: OCT3/4, SOX2, KLF4, C-MYC, LIN28, and NANOG.

20. (canceled)

21. The method of claim 1, wherein the porcine cells comprise germ cells, and wherein the germ cells are combined via IVF to produce an embryo.

22. The method of claim 21, wherein the embryo is transferred to porcine stem cell media between days 1 and 12 post-fertilization.

23. The method of claim 1, wherein the porcine cells comprise cells obtained from non-IVF derived embryos.

24. The method of claim 1, further comprising selecting morphologically stem cell-like colonies from the porcine stem cell media.

25-27. (canceled)

28. The method of claim 1, wherein the porcine pluripotent stem cells are gene-edited.

29. The method of claim 28, wherein the porcine pluripotent stem cells are gene-edited via a CRISPR/Cas9 system, a CRISPR system, a TALEN system, a base editing system, a CRISPR/Cas9-derived RNA-guided engineered nuclease (RGEN) system, a zinc finger nuclease system, a prime editing system, or a variant thereof.

30. The method of claim 28, wherein the porcine pluripotent stem cells are gene-edited at one or more target loci.

31. The method of claim 30, wherein the gene-editing occurs at the one or more target loci simultaneously.

32. The method of claim 31, wherein one, two, three, four, five, six, seven, eight, nine, or ten targe loci are edited simultaneously.

33. The method of claim 30, wherein the gene-editing occurs at one or more target loci in series.

34. The method of claim 33, wherein one, two, three, four, five, six, seven, eight, nine, or ten target loci are edited in series.

35-51. (canceled)

52. A stem cell line produced by the method of claim 1.


SEQUENCE LISTING (9 sequences)

SEQ ID NO: 1 - DNA, 20 bp, synthetic construct
  source 1..20: mol_type = other DNA
agggcaggagtgatgtaact

SEQ ID NO: 2 - DNA, 20 bp, synthetic construct
  source 1..20: mol_type = other DNA
gttgggaggacgaaaatggt

SEQ ID NO: 3 - DNA, 20 bp, synthetic construct
  source 1..20: mol_type = other DNA
gaaagagaaagcgaaccagt

SEQ ID NO: 4 - DNA, 20 bp, synthetic construct
  source 1..20: mol_type = other DNA
aactacaacagccacaacgt

SEQ ID NO: 5 - DNA, 20 bp, synthetic construct
  source 1..20: mol_type = other DNA
gttcaacgatctcctggacc

SEQ ID NO: 6 - DNA, 20 bp, synthetic construct
  source 1..20: mol_type = other DNA
aaacgcagatccaaaggaga

SEQ ID NO: 7 - DNA, 20 bp, synthetic construct
  source 1..20: mol_type = other DNA
agggagatccgactcgtctg

SEQ ID NO: 8 - DNA, 20 bp, synthetic construct
  source 1..20: mol_type = other DNA
gccatcactgccacccagaa

SEQ ID NO: 9 - DNA, 20 bp, synthetic construct
  source 1..20: mol_type = other DNA
gccagtgagcttcccgttga